(19) United States
(12) Patent Application Publication
(10) Pub. No.: US 2013/0346050 A1
(43) Pub. Date: Dec. 26, 2013

(54) METHOD AND APPARATUS FOR DETERMINING FOCUS OF HIGH-INTENSITY FOCUSED ULTRASOUND

(71) Applicant: SAMSUNG ELECTRONICS CO., LTD., Suwon-si (KR)

(72) Inventors: Jung-bae KIM, Hwaseong-si (KR); Young-kyoo HWANG, Seoul (KR); Won-chul BANG, Seongnam-si (KR); Do-kyoon KIM, Seongnam-si (KR)

(73) Assignee: Samsung Electronics Co., Ltd., Suwon-si (KR)

(21) Appl. No.: 13/856,758

(22) Filed: Apr. 4, 2013

(30) Foreign Application Priority Data
Jun. 21, 2012 (KR) 10-2012-0066991

(51) Int. Cl. A61B 19/00

(52) U.S. Cl. CPC A61B 19/50
USPC 703/11

(57) ABSTRACT

A method and apparatus are provided to determine a focus of high-intensity focused ultrasound (HIFU). The method and apparatus include designating an initial location of an observation point on a three-dimensional (3-D) organ model. The method and apparatus also include determining a first location to which the observation point has moved as a result of a change in a form of the 3-D organ model, and transmitting the ultrasound to the observation point. The method and apparatus further determine a displacement of the observation point through a time taken to receive a reflected wave from the observation point, determine a second location of the observation point using the obtained displacement, and process the first and second locations to determine a final location to which the observation point has moved. The method and apparatus include determining the focus of the HIFU based on the determined final location of the observation point.

### CROSS-REFERENCE TO RELATED APPLICATIONS

This application claims the benefit of under 35 U.S.C. §119(a) Korean Patent Application No. 10-2012-0066991, filed on Jun. 21, 2012, in the Korean Intellectual Property Office, the disclosure of which is incorporated herein in its entirety by reference.

### BACKGROUND

1. Field

The following description relates to a method and apparatus to determine a focus of high-intensity focused ultrasound (HIFU).

2. Description of the Related Art

With the rapid development of medical science, treatments have evolved from invasive surgery methods to minimal-invasive surgery methods. Currently, in a development of non-invasive surgery, a gamma knife, a cyber knife, a high-intensity focused ultrasound (HIFU) knife, and the like have appeared. In particular, the HIFU knife uses an ultrasound; thereby making its use harmless to humans and becoming an environmentally friendly method of medical treatment.

A HIFU treatment is a surgery method to remove and treat a tumor by radiating a HIFU to a tumor part (a focused portion) and inducing focal destruction or necrosis of tumor tissue.

A method of removing a lesion using the HIFU treatment can treat without directly incising the human body and; thus, it is widely used. When radiating the HIFU to a lesion from the outside of the human body, a location of the lesion changes due to the activity of the human body. For example, when a patient breathes during surgery, a location of a lesion changes in accord with the patient's breathing. Accordingly, a location (a focus) to which the HIFU is radiated has to change. A method to track a lesion, having a changing location due to internal movement of the human body, and to radiate the location of the lesion with HIFU has been researched.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a method to determine a focus of high-intensity focused ultrasound (HIFU). The method includes designating an initial location of an observation point on a three-dimensional (3-D) organ model; determining a first location to which the observation point has moved as a result of a change in a form of the 3-D organ model; transmitting the ultrasound to the observation point; determining a displacement of the observation point through a time taken to receive a reflected wave from the observation point; determining a second location of the observation point using the obtained displacement; processing the first and second locations to determine a final location to which the observation point has moved; and determining the focus of the HIFU based on the determined final location of the observation point.

The observation point is a reference point for transmission, the 3-D organ model includes anatomical information of an organ, and the second location is a moved location of the observation point.

The processing of the first and second locations to determine the final location to which the observation point has moved includes assigning respective weights to the first and second locations; adding the weighted first and second locations to determine a location of the observation point of the HIFU; and determining the focus of the HIFU based on the determined location of the observation point in the form of the 3-D organ model.

When a difference between the first location and the initial location of the observation point is larger than a predetermined first critical value, the processing of the first and second locations excludes the first location to determine the final location to which the observation point has moved.

When a difference between the second location and the initial location of the observation point is larger than a predetermined second critical value, the processing of the first and second locations excludes the second location to determine the final location to which the observation point has moved.

The determining of the first location includes generating the 3-D organ model based on medical images of the organ; and transforming the 3-D organ model by comparing a plurality of images with the 3-D model, wherein the plurality of images includes a change of a form of the organ due to activity of a body of a patient.

The generating of the 3-D organ model includes extracting location information about a boundary and internal structure of the organ from the medical images; designating locations of landmark points in the location information; and generating a statistical external appearance model.

The generating of the 3-D organ model further includes transforming the statistical external appearance model into a model reflecting a shape characteristic of the organ of the patient.

The generating of the 3-D organ model includes reflecting the shape characteristic of the organ of the patient in the medical image of the patient.

The determining of the displacement includes determining time differences between times taken to receive ultrasounds transmitted from three or more different points of an ultrasound generating apparatus to the observation point.

In another general aspect, there is provided an apparatus to determine a focus of high-intensity focused ultrasound (HIFU). The apparatus includes a first observation point obtainment unit configured to designate an initial location of an observation point on a three-dimensional (3-D) organ model and configured to determine a first location to which the observation point has moved as a result of a change in a form of the 3-D organ model. The apparatus also includes a second observation point obtainment unit configured to transmit the ultrasound to the observation point, configured to determine a displacement of the observation point through a time taken to receive a reflected wave from the observation point, and configured to determine a second location of the observation point using the obtained displacement. The apparatus includes a determination unit configured to process the first and second locations to determine a final location to which the observation point has moved and configured to determine the focus of the HIFU based on the determined final location of the observation point.

The observation point is a reference point for transmission, the 3-D organ model includes anatomical information of an organ, and the second location is a moved location of the observation point.

The determination unit is further configured to assign respective weights to the first and second locations, configured to add the weighted first and second locations to determine a location of the observation point of the HIFU, and configured to determine the focus of the HIFU based on the determined location of the observation point in the form of the 3-D organ model.

When a difference between the first location and the initial location of the observation point is larger than a predetermined first critical value, the determination unit is further configured to exclude the first location to determine the final location to which the observation point has moved.

When a difference between the second location and the initial location of the observation point is larger than a predetermined second critical value, the determination unit is further configured to exclude the second location to determine the final location to which the observation point has moved.

The first observation point obtainment unit is further configured to generate the 3-D organ model based on medical images indicating the organ and transforms the 3-D organ model by comparing a plurality of images with the 3-D model, wherein the plurality of images includes a change of a form of the organ due to the activity of a body of a patient.

The first observation point obtainment unit is further configured to extract location information about a boundary and internal structure of the organ from the medical images, designate locations of landmark points in the location information, and generate a statistical external appearance model.

The first observation point obtainment unit is further configured to transform the statistical external appearance model into a model reflecting a shape characteristic of the organ of the patient.

The first observation point obtainment unit is further configured to reflect the shape characteristic of the organ of the patient in the medical image of the patient.

The second observation point obtainment unit is configured to determine the displacement based on time differences between times taken to receive ultrasounds transmitted from three or more different points of an ultrasound generating apparatus to the observation point.

In one general aspect, there is provided a non-transitory computer-readable recording medium having recorded thereon a program for executing the method as described above.

Other features and aspects may be apparent from the following detailed description, the drawings, and the claims.

The examples of method and apparatus described enable, at least, to accurately determine a focus of high-intensity focused ultrasound (HIFU), which is changed according to the activity of a target body, by determining a final location of an observation point based on a moved location of the observation point due to a change of a form of a 3-dimensional organ model and a moved location of the observation point through transmission and reception of ultrasound.

### BRIEF DESCRIPTION OF THE DRAWINGS

These and/or other aspects will become apparent and more readily appreciated from the following description of the embodiments, taken in conjunction with the accompanying drawings in which:

FIG. 1 is a schematic diagram of a high-intensity focused ultrasound (HIFU) system, according to an embodiment;

FIG. 2 is a block diagram illustrating a focus determination apparatus as shown in FIG. 1;

FIG. 3 is a diagram illustrating a process of determining a focus of HIFU;

FIG. 4 is a diagram illustrating a process of determining the focus of HIFU;

FIG. 5 is a diagram illustrating a process of determining the focus of HIFU;

FIG. 6 is a diagram illustrating a process of obtaining a changed location of an observation point using transmission and reception of ultrasound;

FIG. 7 is a block diagram illustrating a configuration of an image matching device;

FIG. 8 is a diagram illustrating a process performed by an average model generation unit to extract location coordinate information of a boundary and internal structure of an organ;

FIG. 9 is a flowchart illustrating a process in which an image matching unit fits a private model that includes a reflected transformation of an organ, to a location of the organ in an ultrasound image;

FIG. 10 illustrates a process to obtain an affine transformation function in a two-dimensional (2-D) image;

FIG. 11 illustrates a process to compare an image via an image matching unit;

FIG. 12 is a graph illustrating an up and down movement of an absolute location of a diaphragm; and

FIG. 13 is a diagram illustrating a process of generating a three-dimensional (3-D) organ model that is changed according to the activity of a target body.

### DETAILED DESCRIPTION

The following detailed description is provided to assist the reader in gaining a comprehensive understanding of the methods, apparatuses, and/or systems described herein. Accordingly, various changes, modifications, and equivalents of the methods, apparatuses, and/or systems described herein will be suggested to those of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, description of well-known functions and constructions may be omitted for increased clarity and conciseness.

The units and apparatuses described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

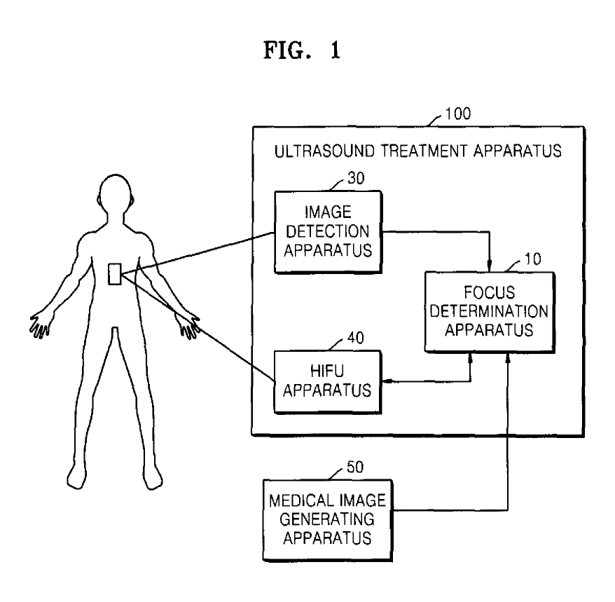

FIG. 1 is a schematic diagram of a high-intensity focused ultrasound (HIFU) system according to an embodiment.

Referring to FIG. 1, the HIFU system includes an ultrasound treatment apparatus 100 and a medical image generating apparatus 50. The ultrasound treatment apparatus 100 includes an image detection apparatus 30, a HIFU apparatus 40, and a focus determination apparatus 10.

The ultrasound treatment apparatus 100 is an apparatus for removing a lesion by radiating HIFU to the lesion of a target body. The ultrasound treatment apparatus 100 determines a focus of the HIFU, in real time, to accurately radiate the HIFU to a lesion that changes location due to a movement of a target body. Accordingly, although a location of a lesion changes due to a movement of the target body, the ultrasound treatment apparatus 100 may accurately radiate HIFU to the lesion that has changed location.

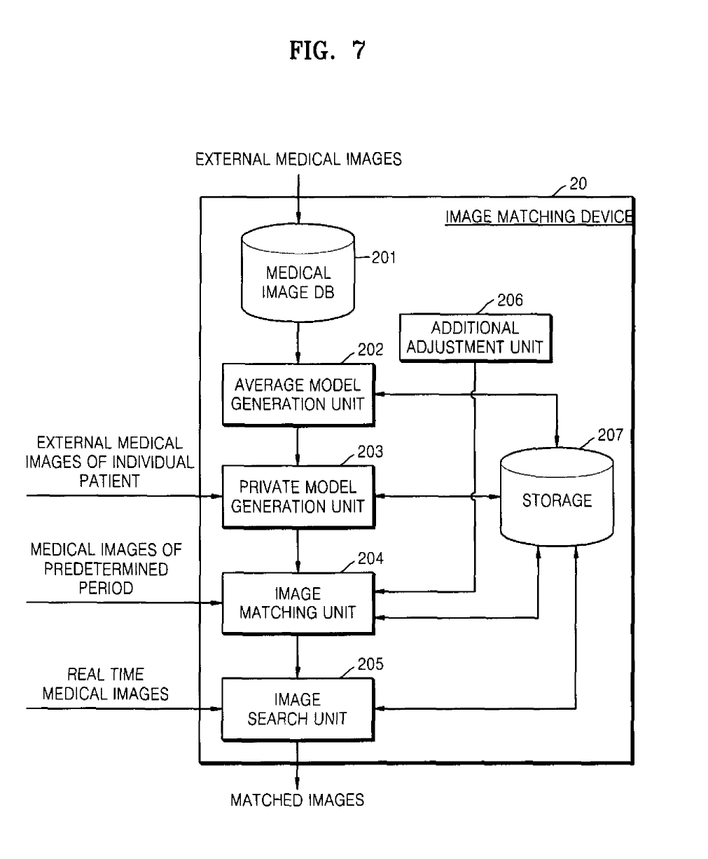

The ultrasound treatment apparatus 100 obtains images of an organ, which repositions or changes location due to a movement of a target body, by using a three-dimensional (3-D) organ model of the organ. The ultrasound treatment apparatus 100 tracks a location of a lesion in the organ, in real time, based on the obtained images of the organ. Accordingly, the ultrasound treatment apparatus 100 may target and remove the lesion by radiating HIFU while tracking the lesion. A method in which the ultrasound treatment apparatus 100 uses a 3-D organ model will be described in detail with reference to FIGS. 7 through 13.

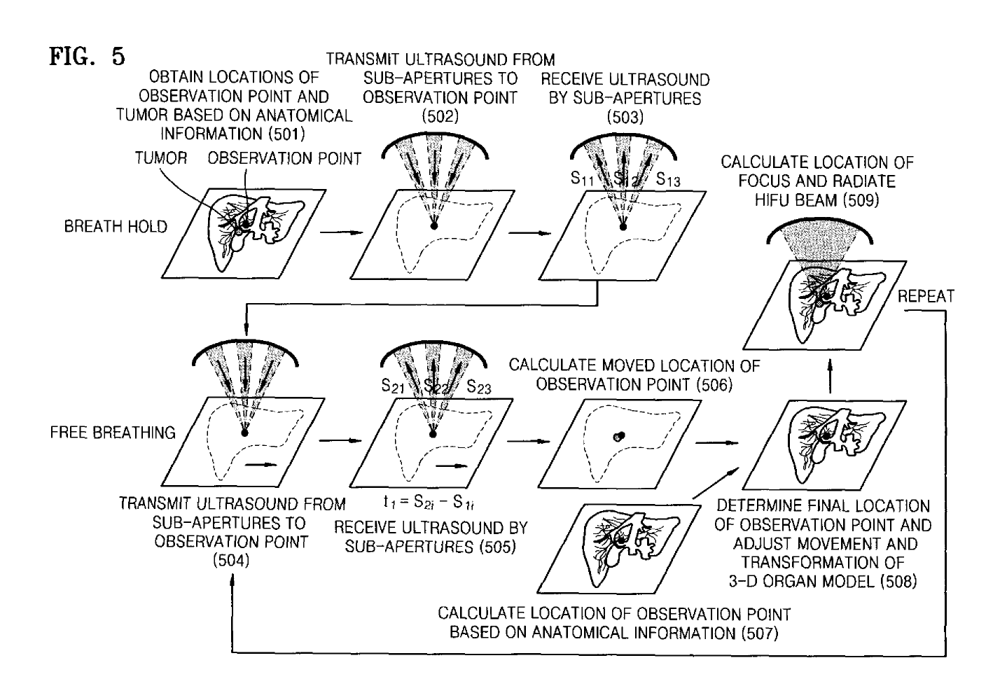

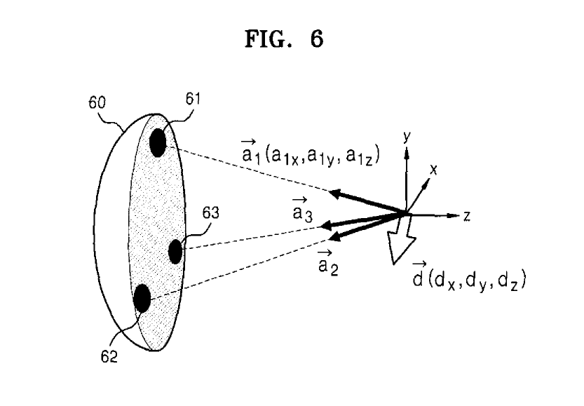

To track the lesion, the ultrasound treatment apparatus 100 measures the time taken until receiving reflected waves after transmitting ultrasounds from sub-apertures of the HIFU apparatus 40 and determines a location of a lesion using the measured time. For instance, in a case that the HIFU apparatus 40 has three or more sub-apertures, the three or more sub-apertures transmit or receive ultrasounds at different locations of the HIFU apparatus 40, the times taken until the sub-apertures receive respective reflected waves are different from each other according to a moving direction of a lesion. The time differences may be use to measure a 3-D moving direction of the lesion. Because the ultrasound treatment apparatus 100 may directly measure a movement of a lesion using ultrasound, the ultrasound treatment apparatus 100 may remove the lesion by radiating HIFU while keeping up with the movement of the lesion. A method in which the ultrasound treatment apparatus 100 tracks a movement of a lesion by using ultrasound will be described in detail with reference to FIGS. 5 and 6.

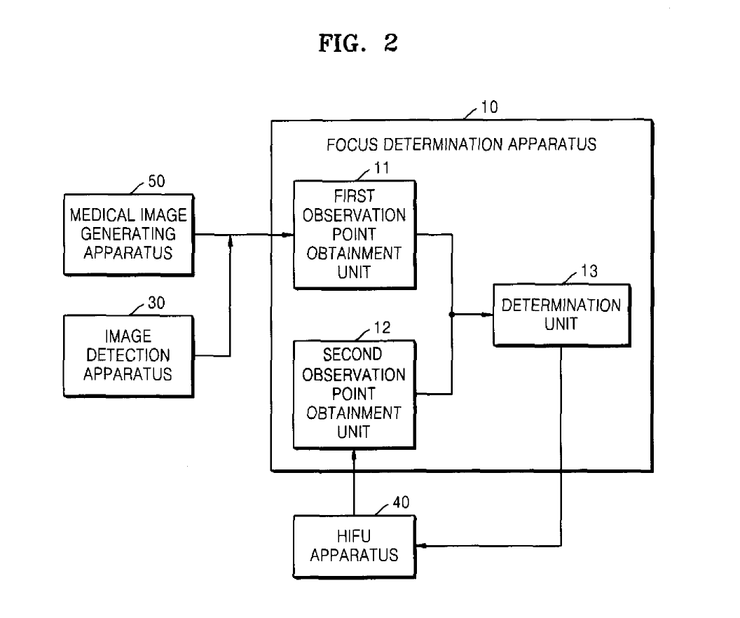

The ultrasound treatment apparatus 100 may precisely track a location of a lesion through a 3-D organ model that tracks a location of a lesion and transmitting and receiving ultrasound signals to track a location of a lesion by using transmission and reception ultrasound signals. That is, the ultrasound treatment apparatus 100 may more accurately track a location of a lesion when using both, the 3-D organ model and the transmission and reception of the ultrasound signals than when tracking the location of the lesion by using only one of the 3-D organ model or the transmission and reception of the ultrasound signals. In more detail, the ultrasound treatment apparatus 100 may determine a final location of a lesion based on a location of the lesion estimated using a 3-D organ model and a location of the lesion estimated using ultrasound. A method in which the ultrasound treatment apparatus 100 determines a final location of a lesion will be described in detail with reference to FIG. 2.

The image detection apparatus 30 is an apparatus to detect images of a target body in real time. For example, the image detection apparatus 30 detects images of a target body, in real time, by transmitting ultrasound to the target body and then receiving ultrasound (a reflected wave) reflected from the target body. Because the image detection apparatus 30 detects images of a target body in real time, the image detection apparatus 30 may obtain images that vary according to a movement of the target body. For example, in the case of a human body, an organ therein moves or is transformed due to breathing. The image detection apparatus 30 outputs images showing a movement or transformation of an organ to the focus determination apparatus 10 in real time.

The image detection apparatus 30 generates image data through responses that are generated when a source signal, which is generated from an installed probe in the image detection apparatus 30, is transmitted to a specific portion of a patient's body that a medical expert, such as a doctor, desires to diagnose. In this case, the source signal may be any one of various signals, such as ultrasound, X-rays, and the like. The case where the image detection apparatus 30 is an ultrasonography machine detecting 3-D images from a patient's body though ultrasound is described as an example as follows.

A probe of the ultrasonography machine generally is manufactured using a piezoelectric transducer. When ultrasound in the range of 2 MHz to 18 MHz is transmitted from a probe of the image detection apparatus 30 to a specific portion of a patient's body, the ultrasound is partially reflected from strata between various different tissues. In particular, the ultrasound is reflected from tissues or internal body fluids with different densities in a target body, for example, blood cells of blood plasma, small structures of organs, etc. The reflected ultrasound vibrates the piezoelectric transducer of the probe, and the piezoelectric transducer outputs electrical pulses according to vibrations thereof. The electrical pulses are converted into images.

The image detection apparatus 30 may output 3-D images as well as 2-D images. In one illustrative example, the image detection apparatus 30 detects a plurality of cross-sectional images of a specific portion of a patient's body while changing a location and orientation of a probe above the patient's body. Subsequently, the image detection apparatus 30 accumulates the cross-sectional images and generates 3-D volume image data showing 3-dimensionally the specific portion of the patient's body. The method of generating 3-D volume image data performed by the image detection apparatus 30 by accumulating cross-sectional images in this manner is called a multi-planar reconstruction (MPR) method. Images (for example, ultrasound images) that may be obtained by the image detection apparatus 30 may be obtained in real time, but is difficult to identify an outline, internal structure, or lesion of an organ.

The medical image generating apparatus 50 is an apparatus to generate detailed images of a target body. For example, the medical image generating apparatus 50 may be an apparatus to generate computed tomography (CT) images or magnetic resonance (MR) images. That is, the medical image generating apparatus 50 generates images in which an outline, internal structure, or lesion of an organ may be clearly identified. The CT images or the MR images may assist in the location of an organ or the location of a lesion. However, the CT images or the MR images cannot be obtained as real time images because the CT images are obtained by using radiation and, thus, require short time photographing due to a danger to a patient or surgeon of prolonged radiation exposure. The MR images cannot be obtained in real time because it takes a long time to capture them. As a result, the CT images or the MR images make it difficult to detect with accuracy that an organ has transformed or that the location of the organ has changed due to the breathing or movement of the patient.

Accordingly, it is necessary to provide a method and apparatus that may capture images in real time and clearly identifies an outline, internal structure, or lesion of an organ. Thus, in accordance with an illustrative example, a method and an apparatus in which a location or transformation of an organ or lesion may be identified in real time images by comparing images detected in real time from the image detection apparatus 30 to a 3-D organ model using medical images obtained from the medical image generating apparatus 50 will be described with reference to FIGS. 7 through 13 below.

The HIFU apparatus 40 is an apparatus that removes or treats a lesion by radiating HIFU to a focused portion to be treated and induces focal destruction or necrosis of the lesion. If the HIFU apparatus 40 continuously radiates HIFU to a specific location while focusing the HIFU on the specific location, the temperature of cells of the specific location rises and tissues of which a temperature rises over a predetermined temperature are necrosed.

The HIFU apparatus 40 transmits ultrasound to an observation point and receives a reflected wave. A plurality of sub-apertures of the HIFU apparatus 40 transmits the ultrasound to the observation point. In this case, the sub-apertures transmit the ultrasound to the observation point at different times with a time difference and receive respective reflected waves from the observation point. The HIFU apparatus 40 transmits and receives ultrasound when the observation point does not change (when not breathing) and also when the observation point changes (when breathing). A changed location of the observation point may be measured by comparing a time taken for transmitting and receiving ultrasound when the observation point does not change and a time taken for transmitting and receiving ultrasound when the observation point changes. An operation regarding this will be described in detail with reference to FIGS. 5 and 6 below.

The observation point is a point that is defined as a reference point to set a location on which the HIFU apparatus 40 focuses ultrasound. A focus is a point on which ultrasound that is generated by a transducer 60 of the HIFU apparatus 40 is focused. Generally, the focus is a location of a lesion to be removed. In the focus, a change including a rise of temperature of cells and expansion of bulk occurs due to focusing of ultrasound resulting in a change in transmission and reception of ultrasound. Accordingly, it is difficult to confirm whether ultrasound is continuously focused on a predetermined region. To overcome this difficulty, the observation point is set as a place adjacent to the focus. A physical change does not occur in cells located at the observation point because ultrasound is not focused on the observation point. Accordingly, the HIFU apparatus 40 sets a relative location as the focus based on the observation point and focuses ultrasound on the focus. The HIFU apparatus 40 continuously confirms a location of the observation point by transmitting and receiving ultrasound to and from the observation point, sets the focus based on the confirmed observation point, and then focuses ultrasound on the focus.

The HIFU apparatus 40 outputs information about the observation point to the focus determination apparatus 10 and receives information about the focus from the focus determination apparatus 10. The HIFU apparatus 40 outputs information about the observation point and information about a changed location of the observation point to the focus determination apparatus 10. Alternatively, the HIFU apparatus 40 outputs information about a time that the ultrasound is transmitted to the observation point and a time the ultrasound reflected from the observation point is received to the focus determination apparatus 10. That is, by using transmission and reception times of the ultrasound, the HIFU apparatus 40 or the focus determination apparatus 10 obtains a changed location of the observation point. The HIFU apparatus 40 receives a focus determined by the focus determination apparatus 10. The HIFU apparatus 40 removes a lesion by focusing ultrasound on the received focus.

The focus determination apparatus 10 determines a point (focus) on which the HIFU apparatus 40 focuses ultrasound. The focus determination apparatus 10 determines a location of a focus that changed or moved as a result of the activity of a human body and provides the determined location of the focus to the HIFU apparatus 40. The focus determination apparatus 10 determines a first changed location of an observation point through real time images received from the image detection apparatus 30 and medical images received from the medical image generating apparatus 50. The focus determination apparatus 10 determines a second changed location of the observation point received from the HIFU apparatus 40. The focus determination apparatus 10 determines a final changed location of the observation point based on the first and second changed locations. In addition, the focus determination apparatus 10 may determine a location of the observation point of a current time based on the first and second changed locations and a location of the observation point of a previous time. The focus determination apparatus 10 determines a changed focus based on location relations between a determined moved location of the observation point and the focus. In one example, the initial location relations between the observation point and the focus may be previously set or defined. For example, the focus may be set as a location apart from the observation point by a specific distance. The focus determination apparatus 10 outputs a determined focus to the HIFU apparatus 40, and the HIFU apparatus 40 focuses ultrasound to the determined focus.

FIG. 2 is a block diagram illustrating a focus determination apparatus as shown in FIG. 1. Referring to FIG. 2, the focus determination apparatus 10 includes a first observation point obtainment unit 11, a second observation point obtainment unit 12, and a determination unit 13. The focus determination apparatus 10 determines a focus of the HIFU apparatus 40 based on information received from the medical image generating apparatus 50, the image detection apparatus 30, and the HIFU apparatus 40, and outputs the determined focus to the HIFU apparatus 40.

The first observation point obtainment unit 11 obtains a location to which an observation point has moved based on image information input from the medical image generating apparatus 50 and the image detection apparatus 30. Image information input from the medical image generating apparatus 50 includes medical images in which an outline, internal structure, or lesion of an organ may be identified. Image information input from the image detection apparatus 30 includes real time images captured by photographing a target body in real time. The real time images may have lower resolution than the medical images received from the medical image generating apparatus 50. The image detection apparatus 30 provides real time images of an organ captured in real time to the first observation point obtainment unit 11.

The first observation point obtainment unit 11 generates a 3-D organ model by using the medical images from the medical image generating apparatus 50. The 3-D organ model is a shape indicating 3-dimensionally an outline of an organ or a lesion in an organ. In one example, the first observation point obtainment unit 11 generates the 3-D organ model by transforming a model generated based on medical images received from various individuals through medical images received from a specific patient. In addition, the first observation point obtainment unit 11 may generate a 3-D organ model of a specific patient through medical images received from the specific patient.

The first observation point obtainment unit 11 obtains a moved or a changed location of an observation point located in the 3-D organ model by transforming the 3-D organ model. Specifically, the first observation point obtainment unit 11 transforms the 3-D organ model in real time using real time images received from the image detection apparatus 30. The first observation point obtainment unit 11 changes the 3-D organ model by comparing or matching the real time images with the 3-D organ model in real time. The first observation point obtainment unit 11 obtains an updated or new location to which an original location of an observation point in the 3-D organ model moved due to a transformation of the 3-D organ model. Because the observation point is located in a specific point of the 3-D organ model, the location of the observation point also changed when the 3-D organ model is transformed. For example, when the transformed 3-D organ model moves from the existing location to the upper side, the lower side, the left side, the right side, or the like, the location of the observation point also changes according to a movement of the transformed 3-D organ model. Also, when the transformed 3-D organ model is lengthened, shortened, or bent, the location of the observation point also changes according to the transformed 3-D organ model.

The second observation point obtainment unit 12 determines a location of the observation point based on information received from the HIFU apparatus 40. The information received from the HIFU apparatus 40 includes times necessary to obtain the location of the observation point. The second observation point obtainment unit 12 measures a time taken to receive ultrasounds transmitted from, for instance, three or more different locations of the HIFU apparatus 40 to the observation point. Using a triangulation method, the second observation point obtainment unit 12 obtains a moved or changed location of the observation point. The time taken to receive ultrasounds transmitted from the three or more different locations to the observation point varies according to a moving direction of the observation point. For example, when the observation point distances more from one of the locations of the three or more different locations, a time taken to transmit and receive ultrasound at the one location increases compared to times taken to transmit and receive ultrasounds at the other locations. The second observation point obtainment unit 12 determines a moved location of the observation point through the time difference. A method of obtaining a moved location of the observation point through the triangulation method is described in detail with reference to FIGS. 5 and 6 below.

The determination unit 13 determines a location of a focus based on locations of observation points received from the first and second observation point obtainment units 11 and 12. For example, the determination unit 13 separates the case where the locations of the received observation points are the same and the case where the locations of the received observation points are not the same, to determine the location of the focus. When the locations of the received observation points are the same, the determination unit 13 determines the locations of the received observation points as a final location of the observation point and determines the location of the focus based on the final location of the observation point. When the locations of the received observation points are not the same, the determination unit 13 determines any one of the points between the received observation points as the final location of the observation point and determines the location of the focus based on the final location of the observation point. For example, the determination unit 13 determines a final location of an observation point by weight-adding up observation points received according to Equation 1.

Cₜ=wₐAₜ+w_{b}Bₜ (1)

In Equation 1, “Cₜ” is a final location of an observation point at a time t, “Aₜ” is a location of the observation point obtained by the first observation point obtainment unit 11 at a time t, and “Bₜ” is a location of the observation point obtained by the second observation point obtainment unit 12 at a time t. “wₐ” is a confidence value for “Aₜ”, and “w_{b}” is a confidence value for “Bₜ”. The sum of “wₐ” and “w_{b}” is “1”.

As another example, the determination unit 13 determines a location of a focus, based on locations of observation points received from the first and second observation point obtainment units 12 and a final location of an observation point of a previous time. The determination unit 13 refers to a previous location of an observation point when determining a final location of the observation point. For example, the determination unit 13 determines a final location of an observation point by weight-adding up locations of observation points received according to Equation 2 and a final location of the observation point of a previous time.

Cₜ=wₐAₜ+w_{b}Bₜ+w_{c}Cₜ₋₁ (2)

In Equation 2, “Cₜ₋₁” is a final location of an observation point at a time t−1, “w_{c}” is a confidence value for “Cₜ₋₁”, and the sum of “wₐ”, “wb”, and “w_{c}” is “1”. Values of wₐ, wb, and w_{c} may be arbitrarily set by a user. Alternatively, the values of “wₐ”, “w_{b}”, and “w_{c}” may be set to have lower values as “Aₜ”, “Bₜ”, and “Cₜ₋₁” distance more from central points of “Aₜ”, “Bₜ”, and “Cₜ₋₁”, respectively. In addition, the values of “wₐ”, “w_{b}”, and “w_{c}” may be set in consideration of a difference between “At” and “Cₜ₋₁” and a difference between “Bt” and “Cₜ₋₁”. For example, when the difference between “Bₜ” and “Cₜ₋₁” is larger than the difference between “Aₜ” and “Cₜ₋₁”, the value of “w_{b}” may be set to have a value that is smaller than that of “wₐ”. Also, the values of wₐ, w_{b}, and w_{c} may be set by using various methods. If a difference between a location of “Aₜ” and a location “Cₜ₋₁” is larger than a predetermined critical value, the focus determination apparatus 10 excludes “Aₜ” when determining a location of an observation point. If a difference between a location of “Bₜ” and a location of “Cₜ₋₁” is larger than a predetermined critical value, the focus determination apparatus 10 excludes “Bₜ” when determining a location of an observation point. That a difference between a location of “Aₜ” or “Bₜ” and a location of “Cₜ₋₁” is larger than a predetermined critical value indicates that “Aₜ” or “Bₜ” goes beyond error bounds. Accordingly, that a difference between a location of “Aₜ” or “Bt” and a location of “Cₜ₋₁” is larger than a predetermined critical value indicates that “Aₜ” or “Bₜ” is excluded when determining “Cₜ”.

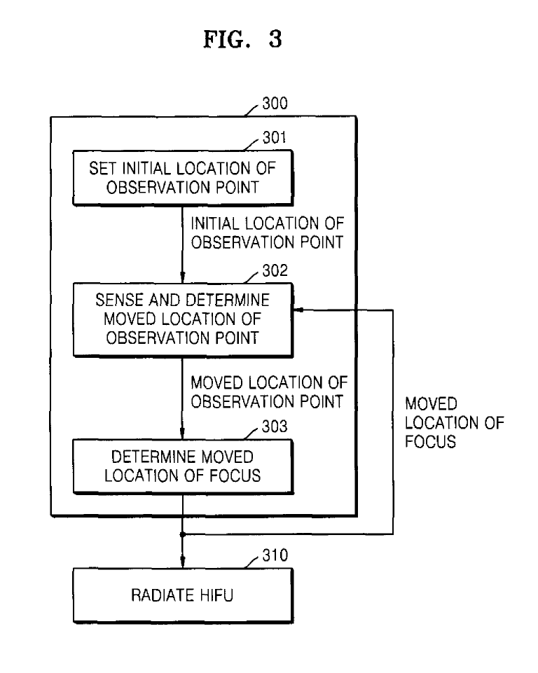

FIG. 3 is a diagram illustrating a process of determining a focus of HIFU. That is, FIG. 3 is a diagram illustrating a process in which the focus determination apparatus 10 of FIG. 2 determines a focus. Accordingly, although omitted, the above descriptions of the focus determination apparatus 10 illustrated in FIG. 2 also apply to an example of FIG. 3.

Referring to FIG. 3, the focus determination apparatus 10 sets an initial location of an observation point (operation 301), senses and calculates a changed or a moved location of the observation point (operation 302), and calculates a changed or moved location of a focus (operation 303). The focus determination apparatus 10 outputs the calculated moved location of the focus to the HIFU apparatus 40. The calculated moved location of the focus is considered when calculating a moved location of a next focus. The HIFU apparatus 40 radiates HIFU to a received moved location of the focus (operation 310). The focus determination apparatus 10 sets the initial location of the observation point at a location adjacent to the focus. The focus determination apparatus 10 senses a moved location of an observation point through the triangulation method and determines the moved location of the observation point considering a moved location of an observation obtained by using a 3-D organ model. The focus determination apparatus 10 determines the moved location of a focus according to the determined moved location of the observation point. In other words, the focus determination apparatus 10 determines a point apart from the moved location of the observation point by a predetermined distance as the focus based on a location relation between the observation point and the focus. The determination unit 13 outputs the moved location of the focus to the HIFU apparatus 40 and refers to the moved location of the focal location when determining a moved location of a next observation point.

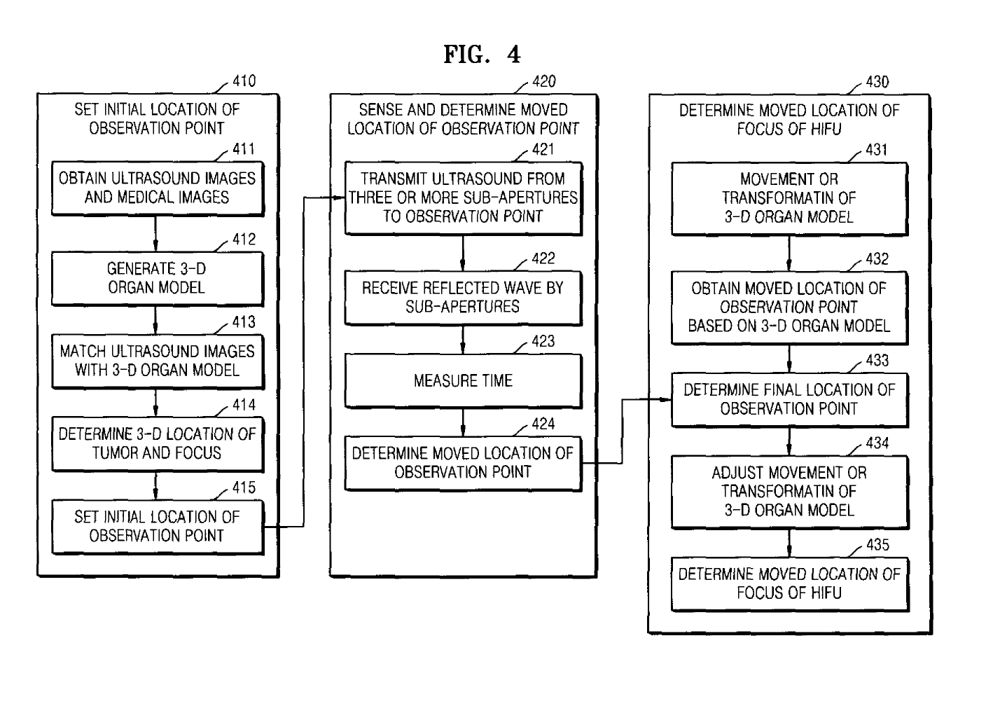

FIG. 4 is a diagram illustrating a process of determining the focus of HIFU. That is, FIG. 4 is a diagram illustrating a process in which the focus determination apparatus 10 of FIG. 2 determines a focus. Accordingly, although omitted, the above descriptions of the focus determination apparatus 10 illustrated in FIG. 2 also apply to FIG. 4.

An operation in which the focus determination apparatus 10 calculates a moved location of a focus is divided into three operations. A first operation is an operation in which the focus determination apparatus 10 sets an initial location of an observation point (operation 410), a second operation is an operation of sensing a changed or moved location of the observation point and determining the moved location of the observation point (operation 420), and a third operation is an operation of determining a changed or moved location of a focus of HIFU (operation 430).

With respect to an operation of setting an initial location of an observation point, the focus determination apparatus 10 obtains ultrasound images and medical images (operation 411) and generates a 3-D organ model by using the obtained ultrasound images and medical images (operation 412). The focus determination apparatus 10 obtains a transformed 3-D organ model by comparing or matching the obtained ultrasound images with the 3-D organ model (operation 413). The focus determination apparatus 10 determines a 3-D location of a lesion, such as a tumor, in the obtained transformed 3-D model and determines the 3-D location of the lesion as an initial location of a focus (operation 414). The focus determination apparatus 10 sets a point adjacent to the determined initial location of the focus as an observation point (operation 415). The set observation point also is a point of the 3-D organ model.

With respect to an operation of sensing and determining a moved location of the observation point, the sub-apertures of the HIFU apparatus 40 transmit ultrasound to the observation point (operation 421) and receive a reflected wave from the observation point (operation 422). The HIFU apparatus 40 directly measures a transmission and reception time of the ultrasound with respect to the observation point (operation 423) and outputs the measured time to the focus determination apparatus 10. In addition, the HIFU apparatus 40 may output a time when the HIFU apparatus 40 transmits the ultrasound to the observation point and a time when the HIFU apparatus 40 receives a reflected ultrasound from the observation point to the focus determination apparatus 10. In this case, the focus determination apparatus 10 calculates a transmission and reception time of the ultrasound with respect to the observation point. The focus determination apparatus 10 determines a moved location of the observation point using the transmission and reception time of the ultrasound with respect to the observation point (operation 424).

With respect to an operation of determining a moved location of a focus of HIFU, the focus determination apparatus 10 obtains a changed or a moved location of the observation point based on a 3-D organ model through a change, movement, or transformation of the 3-D organ model (operations 431 and 432). The focus determination apparatus 10 determines a final location of the observation point based on the moved location of the observation point, which has been determined in the operation of sensing and determining, and the moved location of the observation point, which has been obtained based on the 3-D organ model (operation 433). The focus determination apparatus 10 adjusts a movement or transformation of the 3-D organ model based on the determined final location of the observation point (operation 434). The focus determination apparatus 10 determines a moved location of a focus based on the determined final location of the observation point (operation 435). The movement or transformation of the 3-D organ model is performed through a comparison between real time images that are received in real time and the 3-D organ model. When a final location of the observation point is determined, the movement or transformation of the 3-D organ model is adjusted. In other words, the movement or a transformation of the 3-D organ model is performed through a comparison with real time images and is adjusted again in consideration of the final location of the observation point.

FIG. 5 is a diagram illustrating a process of determining the focus of HIFU. Referring to FIG. 5, operations of the focus determination apparatus 10 are described with respect to the case where a target body has stopped breathing (operations 501 through 503) and the case where the target body is breathing (operations 504 through 509). The focus determination apparatus 10 may determine a location of a focus through a time difference between a time between transmission and reception of the ultrasound when the target body has stopped breathing and a time between transmission and reception of ultrasound when the target body is breathing.

When the target body has stopped breathing, the focus determination apparatus 10 obtains a location of an observation point and a location of a lesion, such as a tumor, and measures a time taken to transmit ultrasound to the observation point and then receive a reflected wave from the observation point.

In operation 501, the focus determination apparatus 10 obtains the location of the observation point and the location of the tumor based on anatomical information of an organ. The anatomical information of the organ is obtained through computed tomography (CT) or magnetic resonance imaging (MRI) images. In other words, the focus determination apparatus 10 determines a location point that the tumor is located in a 3-D organ model generated through CT or MR images and designates any point adjacent to the tumor as an initial location of the observation point. For example, the HIFU apparatus 40 sets a location of a focus as a location that is the same as the location of the tumor.

In operation 502, the sub-apertures of the HIFU apparatus 40 transmit ultrasound to the observation point. The number of sub-apertures may be more than three, and the sub-apertures may be located at different points. In operation 503, the sub-apertures receive reflected waves S11, S12, and S13 that are reflected from the observation point. The HIFU apparatus 40 measures the durations of time taken to transmit and receive ultrasounds to and from the sub-apertures. In a non-breathing state, the observation point hardly moves during transmission and reception of the ultrasounds. The reflected waves S11, S12, and S13 are ultrasounds that are received in three sub-apertures, respectively. The HIFU apparatus 40 outputs the measured times to the focus determination apparatus 10.

In a breathing state, the focus determination apparatus 10 determines a location of the observation point based on the times taken to transmit and receive ultrasound and the anatomical information. In the case that the patient is not breathing, the focus determination apparatus 10 obtains a location of the observation point by comparing a time taken during transmission and reception of ultrasound in a non-breathing state with a time taken during transmission and reception of ultrasound in a breathing state. In operations 504 and 505, the HIFU apparatus 40 measures the durations of time taken to transmit ultrasounds from three or more sub-apertures to the observation point and then receive the reflected waves S21, S22, and S23 from the observation point by the sub-apertures. The measured durations of time are provided to the focus determination apparatus 10.

In operation 506, the focus determination apparatus 10 obtains a location of the observation point by using differences between the measured durations of time. In other words, the focus determination apparatus 10 calculates a difference between a time measured during breathing and a time measured when not breathing in each of the sub-apertures. The observation point does not move when the patient is not breathing, but moves while the patient is breathing. Accordingly, during breathing, a location of the observation point during transmission of the ultrasound and a location of the observation point during reception of a reflected wave differ from each other. A path difference of an ultrasound occurs by a movement distance of the observation point and, thus, a transmission and reception time of the ultrasound is prolonged due to the path difference. The focus determination apparatus 10 obtains a location of the observation point through the triangulation method using differences between durations of time measured in the three or more apertures. A method in which the focus determination apparatus 10 obtains a moved location of the observation point by using the triangulation method is described in detail with reference to FIG. 6 below.

In operation 507, the focus determination apparatus 10 obtains a location of the observation point based on anatomical information. The focus determination apparatus 10 generates a 3-D organ model and obtains a moved location of the observation point located in a 3-D organ model by moving and transforming the 3-D organ model through a comparison between ultrasound images received from the image detection apparatus 30 and the 3-D organ model. In other words, the focus determination apparatus 10 may designate a location of the observation point on the 3-D organ model and may determine whether the designated location of the observation point moves towards another point due to a movement and transformation of the 3-D organ model.

In operation 508, the focus determination apparatus 10 determines a final location of the observation point based on the locations of the observation points obtained in operations 506 and 507 and adjusts a movement and transformation of the 3-D organ model. A method in which the focus determination apparatus 10 determines a final location of the observation point based on the locations of the observation points obtained in operations 506 and 507 has been described in detail with reference to Equations 1 and 2. The focus determination apparatus 10 moves and transforms a 3-D organ model based on the determined final location of the observation point. In other words, the 3-D organ model moves and transforms through a comparison between the received ultrasound images, and when a final location of the observation point is determined, the focus determination apparatus 10 finally adjusts the 3-D organ model based on the final location of the observation point.

In operation 509, the HIFU apparatus 40 radiates HIFU on a location of a focus calculated based on the final location of the observation point. FIG. 6 is a diagram illustrating a process in which the focus determination apparatus 10 calculates displacement of an observation point using the triangulation method. Referring to FIG. 6, three sub-apertures 61 through 63 in a transducer 60 of the HIFU apparatus 40 transmit ultrasound to the observation point and receive reflected waves. The origin of coordinate axes is set as the observation point.

A displacement vector “d” of the observation point is calculated according to Equation 3.

$$d = \frac{c}{2} {( {A}^{T} A )}^{- 1} t \\ In Equation 3 , \\ [ \begin{matrix}{a}_{1 x} {a}_{1 y} {a}_{1 z} \\ {a}_{2 x} {a}_{2 y} {a}_{2 z} \\ … \\ {a}_{Nx} {a}_{Ny} {a}_{Nz}\end{matrix} ] , \\ t = {( {t}_{1} , {t}_{2} , … , {t}_{N} )}^{T} , \\ and \\ d = ( {d}_{x} , {d}_{y} {d}_{z} ) \qquad ( 3 )$$

“c” is a velocity of ultrasound inside the body. “tᵢ” is a time difference measured in each sub-aperture and is calculated by using Equation 4. “aᵢ” is a normalized vector facing an i-th sub-aperture at an observation point and indicates a direction of the ultrasound in the i-th sub-aperture. “aᵢ” is formed of (aᵢₓ, a_{iy}, a_{iz}).

$${t}_{i} = 2 \frac{{a}_{ix} dx + {a}_{iy} dy + {a}_{iz} dz}{c} \qquad ( 4 )$$

When N is 3 (that is, i is 1, 2, or 3), “d” is calculated as follows: The focus determination apparatus 10 calculates time differences t₁, t₂, and t₃ based on information received from the three sub-apertures 61 through 63. Each of the time differences indicates a difference between a time measured when not breathing and a time measured while breathing. Since “a,” is the normalized vector facing the i-th sub-aperture at the observation point, “a,” is determined according to locations of the observation point and sub-apertures before movement. As stated above, “c” is a velocity of ultrasound inside the body. Accordingly, three simultaneous equations may be obtained by using dx, dy, and dz as variables. d(dx, dy, dz) may be obtained by solving the three simultaneous equations. The focus determination apparatus 10 obtains the current location of the observation point by adding the displacement vector “d” to a previous location of the observation point.

FIGS. 7 through 13 are diagrams describing an apparatus and process of comparing ultrasound images with a 3-D organ model, according to some embodiments. A method of generating a 3-D organ model or a method of comparing ultrasound images with a 3-D organ model is not limited to methods described below and various different methods may exist.

FIG. 7 is a block diagram illustrating a configuration of an image matching device 20. Referring to FIG. 7, the image matching device 20 includes a medical image database (DB) 201, an average model generation unit 202, a private model generation unit 203, an image matching unit 204, an image search unit 205, an additional adjustment unit 206, and a storage 207.

The average model generation unit 202 generates and processes an average model of an organ by receiving various medical images of a patient. In one illustrative example, an organ of a patient is traced by using a private model, such as a personalized model of the patient. The average model is generated by the average model generation unit 202 as a preparatory step to generate the private model because characteristics of an organ, such as shape and size, are different for each individual person. As a result, it is necessary to reflect the characteristics of each individual to provide an accurate surgical operation environment. Various pieces of image information of each individual may be used to obtain an accurate average model. In addition, images at various points of breathing may be obtained to reflect a shape or a form of an organ, which is transformed according to the breathing.

In detail, the average model generation unit 202 receives images (hereinafter, referred to as “external medical images”), which a medical expert has captured for diagnosis of a patient, directly from a photographing apparatus or from an image storage medium. Thus, it is desirable to receive images that make it possible to easily analyze outlines of an organ or a lesion or characteristics of the inside of the organ, as the external medical images. For example, CT images or MR images may be input as the external medical images.

The external medical images may be stored in the medical image DB 201, and the average model generation unit 202 may receive the external medical images stored in the medical image DB 201. The medical image DB 201 may store medical images of various individuals, which may be captured by the photographing apparatus or may be input from the image storage medium. When receiving the external medical images from the medical image DB 201, the average model generation unit 202 may receive all or some of the external medical images from the medical image DB 201 depending on a user's selection.

The average model generation unit 202 may apply a 3-D active shape model (ASM) algorithm based on the received external medical images. In order to apply the 3-D ASM algorithm, the average model generation unit 202 extracts shape, size, and anatomic features of an organ from the received external medical images by analyzing the received external medical images and generates an average model of the organ by averaging them. An example of 3-D ASM algorithm is discussed in the paper “The Use of Active Shape Models For Locating Structure in Medical Images,” T. F. Cootes, A. Hill, C. J. Taylor, and J. Haslam, Image and Vision Computing, Vol. 12, No. 6, July 1994, pp. 355-366, the description of which is hereby incorporated by reference. It is possible to obtain an average shape of the organ by applying the 3-D ASM algorithm, and the average shape of the organ may be transformed by modifying variables.

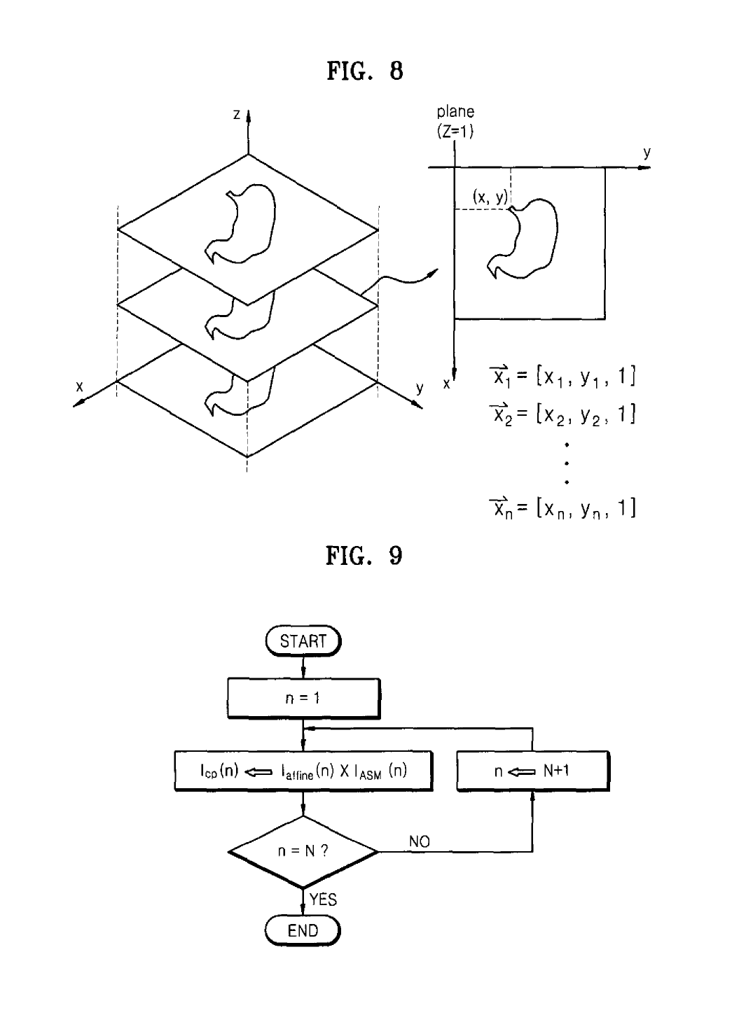

FIG. 8 is a diagram for illustrating a process performed by the average model generation unit 202 to analyze the external medical images. FIG. 8 illustrates a process of extracting location coordinate information of a boundary and internal structure of an organ from the external medical images, for example, the CT or MR images. When the external medical images are input to the average model generation unit 202, the average model generation unit 202 performs an operation of extracting the location coordinate information of the boundary and internal structure of the organ by using different methods depending on whether the external medical images are 2-D images or 3-D images. For example, an internal structure of a liver may include a hepatic artery, a hepatic vein, and a hepatic duct and boundaries between them.

If 2-D images are input as the external medical images, the average model generation unit 202 obtains 3-D volume images showing three-dimensionally of a target part by accumulating a plurality of cross-sectional images to generate a 3-D organ model. This method of obtaining the 3-D volume images is illustrated on the left side of FIG. 8. In more detail, before accumulating the cross-sectional images, the location coordinate information of the boundary and internal structure of the organ is extracted from each of the cross-sectional images. It is then possible to obtain 3-D coordinate information by adding coordinate information of an axis of direction, in which the cross-sectional images are accumulated, to the extracted information. For example, because the image illustrated on the right of FIG. 8 is an image that has a value in the Z-axis of 1, a Z-axis value of a location coordinate value of a boundary extracted from the image is always 1. That is, 3-D coordinate information of the image illustrated on the right of FIG. 8 is [X,Y,1]. As a result, because coordinate information of cross-sections of images illustrated on the left of FIG. 8 is 2-D coordinate information [X,Y], both a coordinate value of the Z-axis and the 2-D coordinate information [X,Y] are extracted to obtain the location coordinate information of the images illustrated on the left of FIG. 8. The location coordinate information of the images may be 3-D coordinate information [X,Y,Z]. If 3-D images are input as the external medical images, cross-sections of the 3-D images are extracted at predetermined intervals and the same process as the case where 2-D images are input as the external medical images is performed, thereby obtaining 3-D location coordinate information. In this process, location coordinate information of a boundary of an organ in 2-D images may be automatically or semi-automatically obtained through an algorithm and may also be manually input by a user with reference to output image information.

For example, in a method of automatically obtaining the location coordinate information of the boundary of the organ, it is possible to obtain location coordinate information of a part in which the brightness of an image is abruptly changed. It is also possible to extract a location of which a frequency value is largest, as a boundary location using a discrete time Fourier transform (DTFT). In a method of semi-automatically obtaining the location coordinate information of the boundary of the organ, when information about a boundary point of an image is input by a user, it is possible to extract the location coordinate of a boundary based on the boundary point, similar to the method of automatically obtaining the location coordinate information. As a result, because the boundary of the organ is continuous and has a looped curve shape, it is possible to obtain information about the entire boundary of the organ. The method of semi-automatically obtaining the location coordinate information does not require searching the whole image; thus, it is possible to rapidly obtain a result compared to a method of automatically obtaining the location coordinate information.

In a method of manually obtaining the location coordinate information of the boundary of the organ, a user may directly designate coordinates of a boundary of an organ while viewing the image. At this time, because an interval at which the coordinates of the boundary of the organ is designated may not be continuous, it is possible to continuously extract the boundary of the organ by performing interpolation on discontinuous sections. If the location coordinate information of the organ or a lesion, obtained by using the above methods, is output after setting a brightness value of a voxel corresponding to the location coordinate to a predetermined value, a doctor, technician, or user, for instance, may confirm shapes of the organ or the lesion expressed three-dimensionally and graphically. For example, if a brightness value of boundary coordinates of a target organ is set to a minimum value, namely, a darkest value, an image of the target organ will have a dark form in an output image. If the brightness value of the target organ is set to a medium value between a white color and a black color and the brightness value of a lesion is set to the black color, it is possible to easily distinguish the lesion from the target organ with the naked eye. The location coordinate information of boundaries and internal structures of a plurality of organs, obtained by using the above methods, may be defined as a data set and be used to perform the 3-D ASM algorithm. The 3-D ASM algorithm is explained below.

In order to apply the 3-D ASM algorithm, coordinate axes of location coordinates of the boundaries and internal structures of the plurality of organs are fit to each other. Fitting the coordinate axes to each other means fitting centers of gravities of the plurality of organs to one origin and aligning directions of the plurality of organs. Thereafter, landmark points are determined in the location coordinate information of the boundaries and internal structures of the plurality of organs. The landmark points are basic points used to apply the 3-D ASM algorithm.

The landmark points may be determined as follows. First, points in which a characteristic of a target is distinctly reflected are determined as the landmark points. For example, the points may include division points of blood vessels of a liver, a boundary between the right atrium and the left atrium in the heart, a boundary between a main vein and an outer wall of the heart, and the like.

Second, the highest points or the lowest points of a target in a predetermined coordinate system are determined as the landmark points.

Third, points for interpolating between the first determined points and the second determined points are determined as the landmark points along a boundary and at predetermined intervals.

The determined landmark points may be represented using coordinates of the X and Y axes in two dimensions and may be represented using coordinates of the X, Y, and Z axes in three dimensions. Thus, if coordinates of each of the landmark points are indicated as vectors x₀, x₁, . . . , xₙ₋₁ in three dimensions (here, n is the number of landmark points), the vectors x₀, x₁, . . . xₙ₋₁ may be represented by the following Equation 5:

$${x}_{i 0} = [ {x}_{i 0} , {y}_{i 0} , {z}_{i 0} ] \\ {x}_{i 1} = [ {x}_{i 1} , {y}_{i 1} , {z}_{i 1} ] \\ ⋮ \\ {x}_{in - 1} = [ {x}_{in - 1} , {y}_{in - 1} , {z}_{in - 1} ] \qquad ( 5 )$$

The subscript i indicates location coordinate information of a boundary and internal structure of an organ, obtained in an i-th image. The number of pieces of location coordinate information may be increased in some cases. As a result, the location coordinate information may be represented as a single vector to facilitate a calculation thereof. Then, a landmark point vector, which expresses all the landmark points with a single vector, may be defined by the following Equation 6:

xᵢ=[xᵢ₀,yᵢ₀,zᵢ₀,xᵢ₁,yᵢ₁,zᵢ₁, . . . ,xᵢₙ₋₁,yᵢₙ₋₁,zᵢₙ₋₁]^{T} (6)

The size of the vector xᵢ is 3n×1. If the number of the data set is N, an average of the landmark points for all the data set may be represented as the following Equation 7:

$$\overline{x} = \frac{1}{N} {\sum }_{i = 1}^{N} {x}_{i} \qquad ( 7 )$$

Similarly, the size of the vector x̅ may be 3n×1. The average model generation unit 202 obtains the average x̅ of the landmark points by calculating Equation 7. If a model is generated based on the average x̅ of the landmark points, the model may become an average organ model. The 3-D ASM algorithm may not only generate the average organ model, but may also transform a form of the average organ model only by adjusting a plurality of parameters. Thus, the average model generation unit 202 calculates not only the average organ model but also an equation so that the plurality of parameters may be applied.

An explanation of an equation for applying the parameters may be described as shown in Equation 8. By Equation 8 below, the average x̅ of the landmark points and differences between data may be represented. In Equation 8, the subscript i indicates an i-th image. Thus, Equation 8 indicates a difference between the landmark points of each image and the average of all images.

dxᵢ=xᵢ− x̅ (8)

By using the difference, a covariance matrix for three variables x, y, and z may be defined as Equation 9 below. The reason for obtaining the covariance matrix is to obtain a unit eigenvector for the plurality of parameters to apply the 3-D ASM algorithm.

S=1/NΣᵢ₌₀^{N}dxᵢdxᵢ^{T}, where 3n×3n (9)

If the unit eigenvector of the covariance matrix S is pₖ, the vector pₖ indicates a transformation of a model generated by using the 3-D ASM algorithm. For example, if a parameter b₁ multiplied by a vector p₁ is changed within the range of −2\sqrt{λ₁}≦b₁<2\sqrt{λ₁}, a width of the model may be changed. If a parameter b₂ multiplied by a vector p₂ is changed within the range of −2\sqrt{λ₂}≦b₂<2\sqrt{λ₂}, a height of the model may be changed. The unit eigenvector pₖ of which a size is 3n×1 may be obtained by using Equation 10 as follows:

Spₖ=λₖpₖ (10)

λₖ indicates an eigenvalue. Finally, the landmark point vector x to which the transformation of the model is applied may be calculated by using the average vector x̅ of the landmark points as in the following Equation 11:

x= x̅+Pb (11)

p=(p₁, p₂, . . . , pₜ) indicates t eigenvectors (here, the size of the pₖ is 3n×1 and the size of p is 3n×t.), and b=(b₁, b₂, . . . , bₜ)^{T} indicates a weight of each eigenvector (here, the size of the b is t×1).

The average model generation unit 202 may calculate x̅ (the size thereof is 3n×1), which indicates a form of an average organ model, and the vector p=(p₁, p₂, . . . Pₜ) (the size thereof is in 3n×t), which is used to apply the transformation of the model by using the 3-D ASM algorithm, by using the equations

The private model generation unit 203 receives the average organ model x̅ and the vector p=(p₁, p₂, . . . , pₜ) from the average model generation unit 202 and then generates a private model through parameter processing of the 3-D ASM algorithm. Because shapes and sizes of organs of vary between patients, accuracy may be lowered if the average organ model is used as it is. For example, an organ of a patient may have a longer, wider, thicker, or thinner form compared to organs of other patients. In addition, if an organ of a patient includes a lesion, the private model generation unit 203 may include a location of the lesion to a model of the organ to accurately capture a shape and location of the lesion. Thus, the private model generation unit 203 receives external medical images of an individual patient from an external image photographing apparatus or the storage 207, analyzes a shape, size, and location of an organ of the individual patient, and, if there is a lesion, analyzes a shape, size, and location of the lesion.

The private model generation unit 203 determines weights (the vector b) of eigenvectors of the 3-D ASM algorithm for the individual patient, based on the medical images such as the CT or MR images in which a shape, size, and location of an organ may be clearly captured. Thus, first, the private model generation unit 203 receives the external medical images of the individual patient and obtains location coordinate information of a boundary and internal structure of an organ. In order to obtain the location coordinate information of the boundary and internal structure of the organ, the private model generation unit 203 uses the process of FIG. 8; namely, the process of analyzing the external medical images, which is performed by the average model generation unit 202. Furthermore, by determining coordinate information of the landmark points through a method that is the same as that used when applying the 3-D ASM algorithm, it is possible to obtain the vector x (the size thereof is 3n×1), which is a private landmark point set of the individual patient. An organ model generated based on the vector x may be a private model. If a characteristic (pₖ^{T}pₖ=1) of a reversed function and unit eigenvector is used in Equation 11, Equation 12 below may be obtained. A value of b=(b₁, b₂, . . . bₜ)^{T} is determined by Equation 12.

b=P^{T}(x− x̅) (12)

The vectors x̅ and p determined by the average model generation unit 202 may be stored in the storage 207 as a database of an average model for a target organ, and may be repeatedly used if necessary. In addition, the external medical images of the individual patient, input to the private model generation unit 202, may be additionally used when determining the average model stored in the database during a medical examination and treatment of another patient.

When the image matching unit 204 receives the vectors x, x̅, p, b from the private model generation unit 203, the image matching unit 204 may match the vectors with a patient's medical images received during a predetermined period. This matching signifies that a model using the 3-D ASM algorithm is overlapped with a location of an organ in an ultrasound medical image to output an output image. In detail, the matching signifies that it is possible to replace or overlap pixel or voxel values corresponding to coordinate information of a model formed by the 3-D ASM algorithm with a predetermined brightness. If the replacement operation is performed, an organ part is removed from an original ultrasound medical image and only a private model is output. If the overlap operation is performed, an image, in which the original ultrasound medical image is overlapped with the private model, may be output. The overlapped image may be easily identified with the naked eye by differentiating a color thereof from that of another image. For example, it may be easy to identify a graphic figure with the naked eye by overlapping a private model with a black and white ultrasound image by using a blue color.

The medical images may be images captured in real time and, for example, may be the ultrasound images. The medical images may be 2-D or 3-D images. The predetermined period may be one breathing cycle because a change of an organ also is generated during a breathing cycle of the body. For example, if one breathing cycle of a patient is 5 seconds, ultrasound images having 100 frames may be generated when ultrasound images are generated 20 frames per 1 second.

A process of comparing or matching, which is performed in the image matching unit 204, may be divided into two operations including an operation reflecting a change of an organ due to breathing in a 3-D organ model in ultrasound images input during a predetermined period; and an operation aligning the transformed 3-D organ model to a target organ in the ultrasound images by performing scale control, axis rotation, and axis movement.

The operation of reflecting a change of an organ due to breathing in a 3-D organ model includes, before comparing the ultrasound images with medical images, a value of the vector b, which is a weight of a parameter of the 3-D ASM algorithm, is controlled by obtaining a location and changing an organ for each frame of the ultrasound images. In one illustrative example, a value of the vector b determined at this time does not have a large difference from a value of the vector b determined in the average model generation unit 202. This small difference is because only a change due to the breathing is reflected in the image matching unit 204, and this change due to the breathing is smaller compared to changes in other individuals. Thus, when determining the value of the vector b, a transformation is performed within a predetermined limited range based on the value of the vector b determined in the average model generation unit 202. In addition, a vector b of a previous frame may be reflected in a determination of a vector b of a next frame because there is no large change during a short period between frames as a change of an organ during the breathing is continuous. If the value of the vector b is determined, it is possible to generate a private model, for each frame, in which a modification of an organ is reflected in each ultrasound image by using a calculation of the 3-D ASM algorithm.

FIG. 9 is a flowchart illustrating a process in which the image matching unit 204 fits a private model to a location of an organ in an ultrasound image through rotation, scale control, and parallel displacement. In the private model, a transformation of the organ is reflected for each image. In detail, FIG. 9 is a flowchart illustrating a process of performing one-to-one affine registration for each frame when the vector b is determined. Vector b is a weight value of an eigenvector for each frame. If the number of frames is N and n is a frame number, a one-to-one match is performed from when n is 1 to when n becomes N. An affine transformation function is obtained by performing an iterative closest point (ICP) algorithm for each frame through a landmark point set of an ultrasound image and a landmark point set of a model. A 3-D body organ model image is obtained through the affine transformation function. The ICP algorithm is an algorithm for rotating and parallel displacing other images and controlling scales of the other images based on an image to align a target in a plurality of images. The ICP algorithm is disclosed in detail in “Iterative point matching for registration of free-form curves and surfaces,” Zhengyou Zhang, International Journal of Computer Vision, 13:2, 119-152 (1994), the description of which is hereby incorporated by reference.

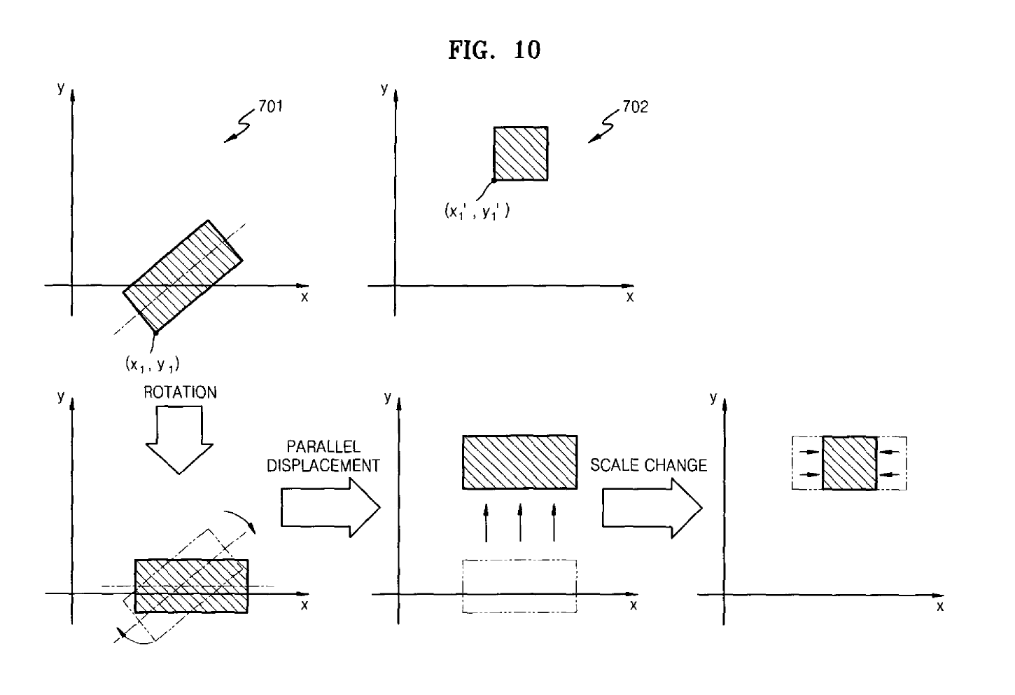

FIG. 10 illustrates a process to obtain the affine transformation function in a 2-D image. A graph 701 illustrates a state before applying the affine transformation, and a graph 702 illustrates a state after applying the affine transformation. Although the rotation, the parallel displacement, and the scale control are performed to apply the transformation, it is possible to determine coefficients of a matrix T_{affine} of the affine transformation function by obtaining first coordinates and final coordinates through the following Equation 13, considering that the affine transformation uses one to one point correspondence.

$$[ \begin{matrix}{x}_{1}^{′} \\ {y}_{1}^{′}\end{matrix} ] = {T}_{affine} [ \begin{matrix}{x}_{1} \\ {y}_{1} \\ 1\end{matrix} ] = [ \begin{matrix}{a}_{1} {b}_{1} {c}_{1} \\ {a}_{2} {b}_{2} {c}_{2}\end{matrix} ] [ \begin{matrix}{x}_{1} \\ {y}_{1} \\ 1\end{matrix} ] \qquad ( 13 )$$

Equation 14 is an equation to apply an affine transformation function obtained in three-dimensions to each frame.

x_{ICP}(n)=T_{affine}(n)×x_{ASM}(n) (14)

Here, n is an integer indicating an n-th frame (1≦n≦N). x_{ASM}(n) indicates a landmark point vector in which the vector b that is the weight value is changed in the image matching unit 204. x_{ICP}(n) includes location coordinate information of organ boundaries and internal structures in which a modification is reflected for each frame. When matching the location coordinate information with the ultrasound image, it is possible to confirm a graphic figure of an organ with the naked eye when a voxel value, corresponding to location coordinates, is replaced or overlapped with a predetermined brightness value in an ulrasonic image.

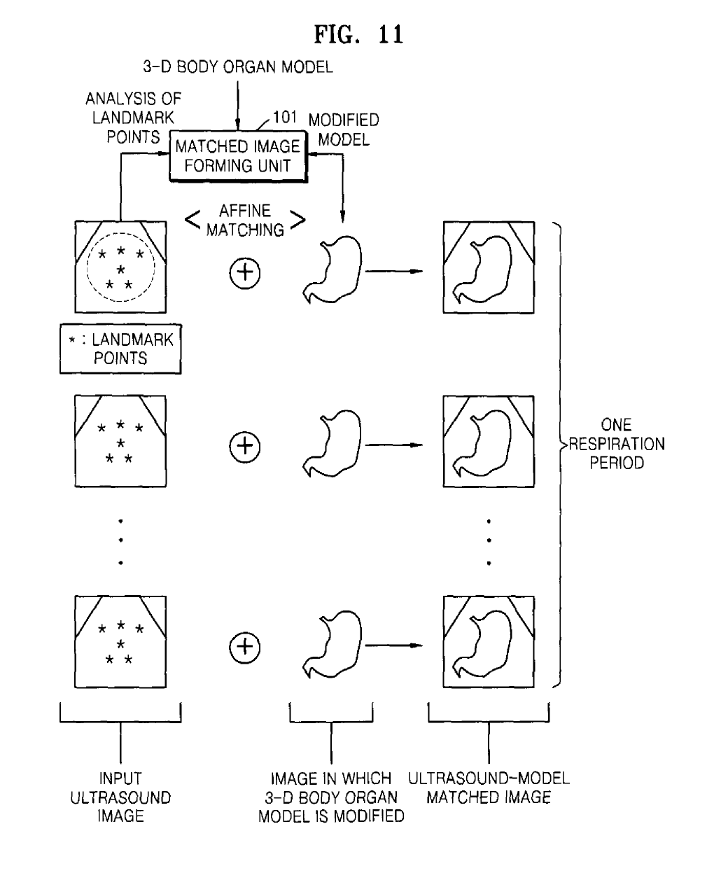

FIG. 11 illustrates a process of comparing or matching an image via the image matching unit 204. FIG. 11 illustrates a process in which the image matching unit 204 generates a matched image between a ultrasound image input during a predetermined period and a body organ model based on an ultrasound image input during one breathing cycle. In FIG. 11, the input ultrasound image is disposed in a left edge portion, and a mark * illustrated in the input ultrasound image indicates a landmark point. The input ultrasound image may reflect various forms of breathing from inspiration to expiration.

A private model generated in the private model generation unit 203 is modified according to breathing. However, a modification according to respiration is smaller than that due to diversity between individuals. Thus, when reflecting a modification according to breathing, it may be faster and easier to adjust parameter values determined by the private model generation unit 203 compared to newly obtained 3-D ASM algorithm. The affine transformation function, T_{affine}, is applied through the ICP algorithm through a landmark point in which the modification has been reflected and a landmark point of an organ of the ultrasound image. Through the affine transformation, a size and location of a 3-D organ model may be modified to match with a size and location of an organ of the ultrasound image. Combining a modified model with the ultrasound image may be performed through a method of replacing or overlapping a pixel or voxel value of the ultrasound image corresponding to a location of a model with a predetermined value. A matched image is referred to as an ultrasound-model matched image and may be stored in the storage 207.

The image search unit 205 performs processes of a surgical operation. In the surgical operation, a graphic shape of an organ is output in an ultrasound image, which is input in real time, on a screen, and then a surgeon performs the surgical operation while confirming the graphic shape of the organ with the naked eye. In accordance with an illustrative configuration, operations of the surgical operation include receiving a real time medical image of a patient. At this time, the real time medical image may be an image which is the same as that received by the image matching unit 204. Thus, for example, if a real time ultrasound image is received, by comparing the real time ultrasound image with medical images input to the image matching unit 204 during a predetermined period, an image that is most similar to the real time ultrasound image is determined. Subsequently, an ultrasound-model matched image corresponding to the determined image is searched in the storage 207, and then a found ultrasound-model matched image is output.

As an example in which the image search unit 205 searches for a similar image in the ultrasound image, a method may be performed to determine an image by detecting a location of a diaphragm. If a location of the diaphragm is X in the real time ultrasound image, the method performs searching for an image having the smallest difference by calculating a difference between the location X and a location of each diaphragm in the medical images input to the image matching unit 204 during the predetermined period.

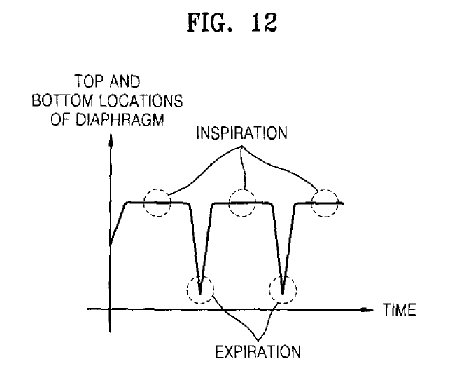

FIG. 12 is a graph illustrating an up and down movement of an absolute location of the diaphragm. As illustrated in the graph, it is possible to confirm that the location of the diaphragm regularly changes in a breathing cycle. A location of a probe and a location of a patient may be fixed when capturing the medical images, which are input to the image matching unit 204 during the predetermined period, and the real time medical images, which are input to the image search unit 205. The reason is that a relative location of an organ in the image may be changed when the location of the probe or the location of the patient changes. It is not possible to accurately and rapidly perform a search operation when comparing images when the relative location changes.

As another example in which the image search unit 205 searches for a similar image in the ultrasound image, a method is provided to determine an image through a brightness difference between pixels. In one illustrative example, the method is configured to consider that a brightness difference between the most similar images is the smallest. In detail, when searching for an image similar to an image (a second image) of a frame of the real time medical image among the medical images (first images) input during the predetermined period to use for comparing or matching, a brightness difference between pixels of one of the first images and pixels of the second image is calculated and then a dispersion for the brightness difference is obtained. Next, brightness differences between pixels of the other images of the first images and pixels of the second image also are calculated and then dispersions for the brightness differences are obtained. Then, an image with the smallest dispersion may be determined as the most similar image.

The additional adjustment unit 206 may output an adjusted final result when a user adjusts the affine transformation function, T_{affine}, and the parameters of the 3-D ASM algorithm while viewing an output image. That is, the user may perform accurate transformation while viewing the output image with the naked eye.

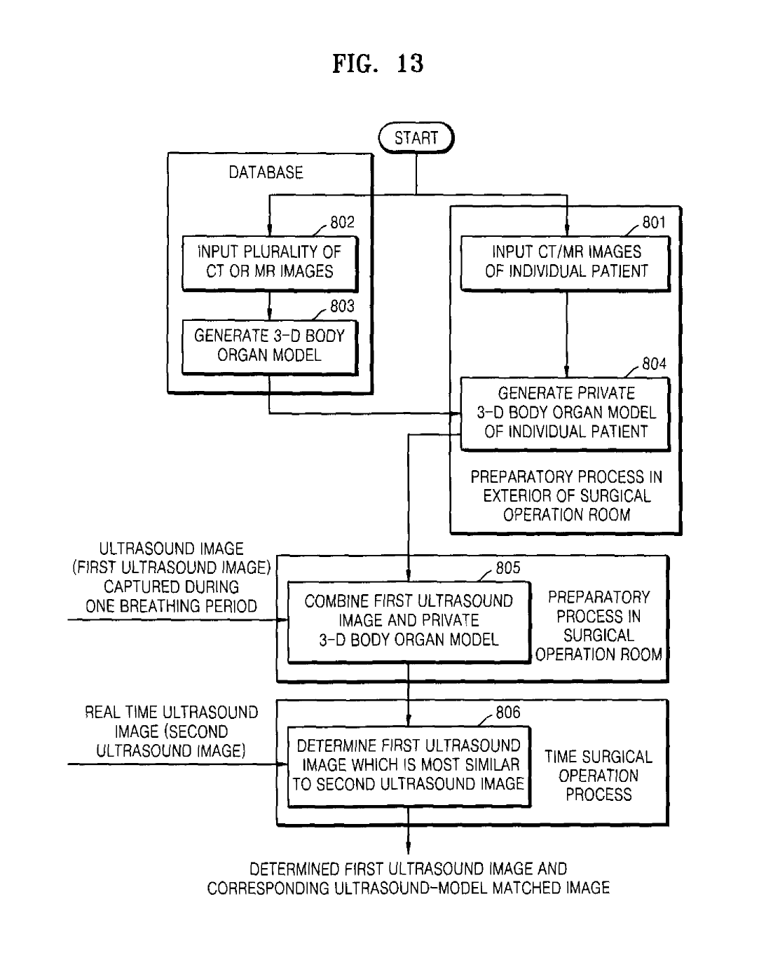

FIG. 13 is a flowchart illustrating a method of tracing a dynamic organ and a lesion based on a 3-D organ model. The results of operations 802 and 803 may be stored in the medical image DB 201 of FIG. 7. In operation 802, CT or MR images of various breathing cycles of individuals are received. In operation 803, a 3-D body organ model is generated based on the received images. At this time, as stated above, the 3-D ASM algorithm may be used.

In operation 801, a CT or MR image of an individual patient is received. In operation 804, the 3-D body organ model generated in operation 803 is modified based on the received image of the individual patient. A process of generating the modified 3-D body organ model, namely, a private 3-D body organ model may be performed outside a surgical operation room as a preparatory process. In operation 805, ultrasound images (first ultrasound images) captured during one breathing cycle of a patient are received, and the first ultrasound images are matched with the private 3-D body organ model. A matched image is referred to as an ultrasound-model matched image and may be stored in a temporary memory or in a storage medium such as a storage. Operation 805 may be performed as a preparatory process in a surgical operation room. In operation 805, a location of the patient may be fixed or established. In addition, in operation 806, a location of a probe may be fixed or established. In operation 806, as a real operation in the surgical operation room, if an ultrasound image (a second ultrasound image) of the patient is input in real time, an image, which is most similar to the second ultrasound image, from among the first ultrasound images is determined. Subsequently, an ultrasound-model matched image corresponding to the determined first ultrasound image is output. The methods according to the above-described embodiments may be recorded, stored, or fixed in one or more non-transitory computer-readable media that includes program instructions to be implemented by a computer to cause a processor to execute or perform the program instructions. The media may also include, alone or in combination with the program instructions, data files, data structures, and the like. The program instructions recorded on the media may be those specially designed and constructed, or they may be of the kind well-known and available to those having skill in the computer software arts. Examples of non-transitory computer-readable media include magnetic media such as hard disks, floppy disks, and magnetic tape; optical media such as CD ROM disks and DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher level code that may be executed by the computer using an interpreter. The described hardware devices may be configured to act as one or more software modules in order to perform the operations and methods described above, or vice versa.

It is to be understood that in the embodiment of the present invention, the operations in FIGS. 2, 3, 4, and 13 are performed in the sequence and manner as shown although the order of some steps and the like may be changed without departing from the spirit and scope of the present invention. In accordance with an illustrative example, a computer program embodied on a non-transitory computer-readable medium may also be provided, encoding instructions to perform at least the method described in FIGS. 2, 3, 4, and 13.

Unless otherwise defined, all terms (including technical and scientific terms) used herein have the same meaning as commonly understood by one of ordinary skill in the art to which the present invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Even after all programs in a computer system including an operating system (OS) are deleted, a first program that is stored in a first storage unit in which a BIOS is stored, is not deleted. Thus, a user may install software automatically and may update software without any difficulty by invoking a second program for installing software automatically and updating software.

While this invention has been particularly shown and described with reference to embodiments thereof, it will be understood by those of ordinary skill in the art that various changes in form and details may be made therein without departing from the spirit and scope of the invention as defined by the appended claims. The embodiments should be considered in a descriptive sense only and not for purposes of limitation. Therefore, the scope of the invention is defined not by the detailed description of the invention but by the appended claims, and all differences within the scope will be construed as being included in the present invention.

What is claimed is:

1. A method to determine a focus of high-intensity focused ultrasound (HIFU), the method comprising:
designating an initial location of an observation point on a three-dimensional (3-D) organ model;
determining a first location to which the observation point has moved as a result of a change in a form of the 3-D organ model;
transmitting the ultrasound to the observation point;
determining a displacement of the observation point through a time taken to receive a reflected wave from the observation point;
determining a second location of the observation point using the obtained displacement;
processing the first and second locations to determine a final location to which the observation point has moved; and
determining the focus of the HIFU based on the determined final location of the observation point.

2. The method of claim 1, wherein the observation point is a reference point for transmission, the 3-D organ model comprises anatomical information of an organ, and the second location is a moved location of the observation point.

3. The method of claim 1, wherein the processing of the first and second locations to determine the final location to which the observation point has moved comprises:
assigning respective weights to the first and second locations;
adding the weighted first and second locations to determine a location of the observation point of the HIFU; and
determining the focus of the HIFU based on the determined location of the observation point in the form of the 3-D organ model.

4. The method of claim 1, wherein when a difference between the first location and the initial location of the observation point is larger than a predetermined first critical value, the processing of the first and second locations excludes the first location to determine the final location to which the observation point has moved.

5. The method of claim 4, wherein when a difference between the second location and the initial location of the observation point is larger than a predetermined second critical value, the processing of the first and second locations excludes the second location to determine the final location to which the observation point has moved.

6. The method of claim 1, wherein the determining of the first location comprises:
generating the 3-D organ model based on medical images of the organ; and
transforming the 3-D organ model by comparing a plurality of images with the 3-D model, wherein the plurality of images comprises a change of a form of the organ due to activity of a body of a patient.

7. The method of claim 6, wherein the generating of the 3-D organ model comprises:
extracting location information about a boundary and internal structure of the organ from the medical images;
designating locations of landmark points in the location information; and
generating a statistical external appearance model.

8. The method of claim 7, wherein the generating of the 3-D organ model further comprises transforming the statistical external appearance model into a model reflecting a shape characteristic of the organ of the patient.

9. The method of claim 8, wherein the generating of the 3-D organ model comprises reflecting the shape characteristic of the organ of the patient in the medical image of the patient.

10. The method of claim 1, wherein the determining of the displacement comprises determining time differences between times taken to receive ultrasounds transmitted from three or more different points of an ultrasound generating apparatus to the observation point.

11. An apparatus to determine a focus of high-intensity focused ultrasound (HIFU), the apparatus comprising:
a first observation point obtainment unit configured to designate an initial location of an observation point on a three-dimensional (3-D) organ model and configured to determine a first location to which the observation point has moved as a result of a change in a form of the 3-D organ model;
a second observation point obtainment unit configured to transmit the ultrasound to the observation point, configured to determine a displacement of the observation point through a time taken to receive a reflected wave from the observation point, and configured to determine a second location of the observation point using the obtained displacement; and
a determination unit configured to process the first and second locations to determine a final location to which the observation point has moved and configured to determine the focus of the HIFU based on the determined final location of the observation point.

12. The apparatus of claim 11, wherein the observation point is a reference point for transmission, the 3-D organ model comprises anatomical information of an organ, and the second location is a moved location of the observation point.

13. The apparatus of claim 11, wherein the determination unit is further configured to assign respective weights to the first and second locations, configured to add the weighted first and second locations to determine a location of the observation point of the HIFU, and configured to determine the focus of the HIFU based on the determined location of the observation point in the form of the 3-D organ model.

14. The apparatus of claim 11, wherein when a difference between the first location and the initial location of the observation point is larger than a predetermined first critical value, the determination unit is further configured to exclude the first location to determine the final location to which the observation point has moved.

15. The apparatus of claim 14, wherein when a difference between the second location and the initial location of the observation point is larger than a predetermined second critical value, the determination unit is further configured to exclude the second location to determine the final location to which the observation point has moved.

16. The apparatus of claim 11, wherein the first observation point obtainment unit is further configured to generate the 3-D organ model based on medical images indicating the organ and transforms the 3-D organ model by comparing a plurality of images with the 3-D model, wherein the plurality of images comprises a change of a form of the organ due to the activity of a body of a patient.

17. The apparatus of claim 16, wherein the first observation point obtainment unit is further configured to extract location information about a boundary and internal structure of the organ from the medical images, designate locations of landmark points in the location information, and generate a statistical external appearance model.

18. The apparatus of claim 17, wherein the first observation point obtainment unit is further configured to transform the statistical external appearance model into a model reflecting a shape characteristic of the organ of the patient.

19. The apparatus of claim 18, wherein the first observation point obtainment unit is further configured to reflect the shape characteristic of the organ of the patient in the medical image of the patient.

20. The apparatus of claim 11, wherein the second observation point obtainment unit is configured to determine the displacement based on time differences between times taken to receive ultrasounds transmitted from three or more different points of an ultrasound generating apparatus to the observation point.

21. A non-transitory computer-readable recording medium having recorded thereon a program for executing the method of claim 1.
